(19) United States
(12) Patent Application Publication
(10) Pub. No.: US 2007/0129745 A1
(43) Pub. Date: Jun. 7, 2007

(54) Acupuncture spring needle with induced magnetic field

(76) Inventors: Jung Suk Suh, Seoul (KR); Kelly Lucas, Wasilla, AK (US); Yong Ki Chong, Renton, WA (US)

Correspondence Address: Eugene Oak, Ph.D., J.D., M.Div.;Law Office of Jane Oak and associates, P.C., Suite #2470, 3435 Wilshire Blvd., Los Angeles, CA, 90010, US

(21) Appl. No.: 11/294,103

(22) Filed: Dec. 6, 2005

(51) Int. Cl. A61B 17/34

(52) U.S. Cl. 606/189

(57) ABSTRACT

A patch type acupuncture needle to facilitate blood circulations in a restricted area of a human body by utilizing an induced magnetic field is provided. The patch type acupuncture induced magnetic needle set of the current application is comprised of; 1) a spring needle of total height 3.1 mm and thickness 0.18 mm, 2) an annular magnet with inner-diameter of 2.2 mm, outer diameter of 7.0 mm, thickness of 2.1 mm and has magnetic strength of 650 Gauss, 3) a circular cap of outer diameter 7.5 mm, inner diameter 7.3 mm and cap wall height 2 mm, and 4) an adhesive plaster which has a round seat of inner-diameter of 7.5 mm for receiving the magnet cap. Half of the spring needle is wound and half is straight. The spring needle is located in the inner hole of the annular magnet. The straight tip of the spring needle protrudes out of the inner hole about 1 mm.

### FIELD OF THE INVENTION

Current application is related with a spring acupuncture needle surrounded by an annular magnetic plate.

### BACKGROUND OF THE INVENTION

Influence of magnetic field on human blood circulation is not widely known to the public. But it is known that human blood is composed of a liquid called plasma, which contains suspended cells of red blood cells, white blood cells, and platelets. Plasma, which is 90% water, typically accounts for 55% by volume of blood and the remaining 45% is from the red blood cells that make up 99% of the cells in the blood. The red blood cells are the principal carriers of the red-colored hemoglobin molecules, an iron-containing protein, and binds about 97% of all oxygen in the body. Dr. Kenneth R. Bridges, M.D. Associate Professor of Medicine at Harvard Medical School reported that the red blood cells change the shape from biconcave to a sickle shape when oxygen is deficient. The normal red cells retain their biconcave shape and move through the smallest capillaries without problem. However, the hemoglobin polymerizes in sickle red cells when it releases oxygen, and deforms the red cells. The membranes of the red blood cells become rigid in part due to repeated episodes of hemoglobin polymerization/depolymerization as the cells pick up and release oxygen in circulation. These rigid red blood cells fail to move through the small blood vessels, blocking local blood flow to a microscopic region of tissue.

Meanwhile, James D. Livingston, Professor of the Department of Materials Science and Engineering at the Massachusetts Institute of Technology, reported the influence of the magnetic field to the human body in terms of increasing blood circulation in Skeptical Inquirer 25-30, 58, 1998. According to him, the human body, like its primary constituent, water, is diamagnetic. In response to an applied magnetic field, the electrons in water molecules make slight adjustments in their motions, producing a net magnetic field in the opposing direction about 100,000 times smaller than the applied field. Some literature suggests that magnetic fields attract blood, citing all the iron it contains. But, the net effect of the weak Para magnetism of the isolated iron atoms in hemoglobin is only a slight decrease in the overall diamagnetism of blood. Blood, like water, is weakly repelled by magnetic fields, not attracted. The results of the Baylor study raise the possibility that at least in some cases, topical application of permanent magnets may indeed be useful in pain relief, a conclusion that should be regarded as tentative until supported by further studies. Any mechanism for such an effect remains mysterious, but an effect of static magnetic fields on the complex electrochemical processes of the human body is not impossible. He guessed that inexpensive refrigerator magnets are as likely to provide help as the more expensive magnets marketed specifically for therapy. The inventor of the current application developed a magnet patch based on an assumption that the polymerized red blood cells align in a row under the influence of a magnetic field and move through the smallest capillaries without problem, alleviating pain.

### DESCRIPTION OF THE PRIOR ARTS

U.S. Pat. No. 6,113,620 to Chung discloses a magnetic needle for acupuncture. The magnetic needle has housing with an opening, and a magnet seated in the opening of the housing. A wedge-shaped projection is held in the opening of the housing so as to project into the exterior of the bottom wall of the housing and come into contact with the magnet. The projection forms an intensive magnetic field around a meridian point having fine electric currents or electromagnetic waves. The projection thus magnetically stimulates the meridian point while performing a magnetic massage effect on the meridian point. The magnetic needle accomplishes an acupuncturing effect for relieving pain and curing disease as expected in typical acupuncture.

But, the tip of the magnetic body cannot penetrate a patient's skin; therefore, the magnetic protrusion is not a needle in terms of traditional Oriental medicine. It is more close to a sharp magnet pointer. However, the magnetic field cannot reach to the pain point of a human body under the skin. Professor James D. Livingston, Department of Materials (MIT) Science and Engineering at the Massachusetts Institute of Technology reported that the electrons in water molecules make slight adjustments in their motions in response to an applied magnetic field, producing a net magnetic field in the opposing direction about 100,000 times smaller than the applied field as reported. Therefore, the effect of the magnetic field of their invention to the human body is doubtful.

U.S. Pat. No. 5,904,700 to Guo illustrates a magnetic apparatus pointer of traditional Chinese medicine for health care, and in particular, relates to a magnetic medicine instrument of acupuncture. The apparatus of pointer includes an air bag, and a permanent magnetic head provided within the hollow case. To operate the pointer of the present invention, the air bag shall be pressed, and at the same time, the hollow case shall be attached closely to the acupuncture point of human body. When the press is released, the air bag restores to the original shape, and the air within the hollow case is sucked into said bag, so as to make the magnet head closely attached to the acupuncture point of human body. The pointers with N pole and S pole may be positioned on both sides of the human body making vertical cutting lines of magnetic force acting upon the human body. This invention is also a magnetic press pointer. Not an acupuncture needle.

U.S. Pat. No. 3,939,841 to Dohring, et al. locating device for transmitting a mild electrical direct current or pulsating direct current through the body of a patient to locate through increased resistance, as indicated on the meter, areas in the body that may benefit from the application of an acupuncture needle. The locating device has an elongated handle with the necessary electrical connection. At one end portion of the handle a transversely formed hole receives an elongated, tubular, electrically conductive needle support and guide. Acupuncture needles are very thin and flexible, and require support to obtain the best usage thereof. This invention is related to the acupuncture needle guide to connect the needle with an electricity source and control the currency.

None of the prior art illustrating an acupuncture needle that is surrounded with a permanent magnet and penetrates the patient's skin to maximize the magnetic field effect to human blood circulation.

### SUMMARY OF THE INVENTION

The inventor of the current application developed a patch type acupuncture needle surrounded by a permanent magnet based on an assumption that the polymerized red blood cells align in a row under the influence of a magnetic field and move through the smallest capillaries without problem, alleviating pain. The patch type acupuncture induced magnetic needle of the current application is comprised of; 1) a spring needle of total height 3.1 mm and thickness 0.18 mm, 2) an annular magnet with inner-diameter of 2.2 mm, outer diameter of 7.0 mm, thickness of 2.1 mm and has magnetic strength of 650 Gauss, 3) a circular cap of outer diameter 7.5 mm, inner diameter 7.3 mm and cap wall height 2 mm, 4) an adhesive plaster which has a round seat of inner-diameter of 7.5 mm for receiving the magnet cap. Half part of the spring needle is wind and half part is straight. The spring needle is located in the inner hole of the annular magnet. The straight tip of the spring needle protrudes out of the inner hole about 1 mm to penetrate into a patient's skin. The protruded tip of the needle is coated with Silver or Gold. The annular magnet is placed in the circular cap. The circular cap is coated with Silver with 0.03 mm thickness. The patch type acupuncture induced magnetic needle of the current application is applied to any painful or bruised point. The skin of the point through where the needle is penetrating should be disinfectionated to prevent contamination by bacteria on the skin. Press the adhesive plaster for 2 to 3 seconds to allow the needle to penetrate the skin. The patch should be replaced by a new one every 24 hours. The effect of the patch type acupuncture induced magnetic needle of the current application disappears when removed from the skin of the patient.

### BRIEF DESCRIPTION OF THE DRAWINGS

FIG. 1 is a cross-sectional view of a patch titled as “Magnetic Needle for Acupuncture” of prior art.

FIG. 2 is a cross-sectional view of a “Magnetic Acupuncture Pointer” of another prior art.

FIG. 3 is an exploded view of an acupuncture spring needle set utilizing induced magnetic field of current application.

FIG. 4 is a cross-sectional view of an acupuncture spring needle set utilizing an induced magnetic field of current application when attached to the skin of a patient.

FIG. 5 is a schematic cross-sectional view comparing the magnetic field effect of the acupuncture spring needle utilizing an induced magnetic field of current application and the magnetic pointer of the prior art when applied to the skin of a patient.

FIG. 6 is a front view of an acupuncture spring needle set utilizing induced magnetic field of current application seen from the view point ‘A’ in FIG. 4.

### DETAILED DESCRIPTION OF THE PREFERRED EMBODIMENT

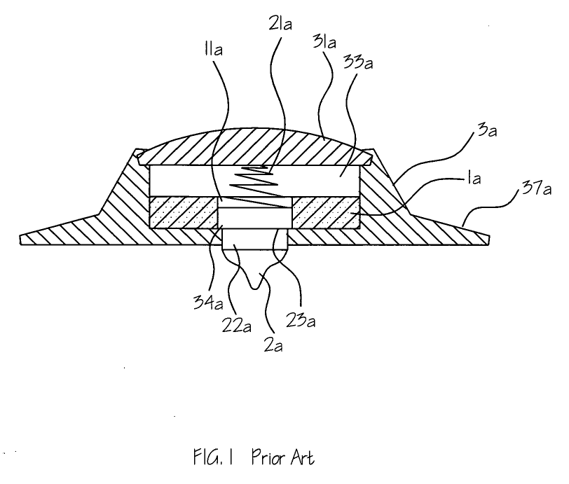

FIG. 1 is a cross-sectional view of a patch titled as “Magnetic Needle for Acupuncture” of prior art. It is described column 3 lines 56 to 58 of the U.S. Pat. No. 6,113,620 that “the magnetic needle is comprised of a magnet 1a, 1b and a magnetically concentrated projection 2a, 2b, with a compression coil spring 21a, 21b being used as the elastic biasing means for the projection.”

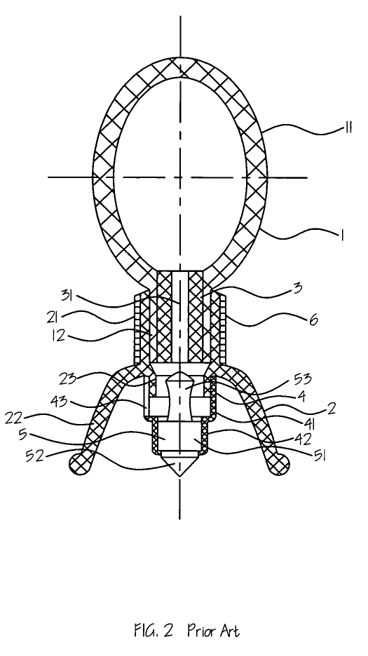

FIG. 2 is a cross-sectional view of a “Magnetic Acupuncture Pointer” of another prior art. In column 3, lines 14 to 19 of the U.S. Pat. No. 5,904,700, it is described that “The permanent magnet 5, fixed within the base 4, has a permanent magnet block 51 and magnet head 52. On the top of said block 51 there is provided with a magnetic yoke 53, and said permanent magnet block 51 has its bottom attached with a magnet head 52 made of metal materials with good magnetic conductivity.” Guo did not use the terminology ‘acupuncture needle’. Instead he used ‘magnetic head 52’. In column 1, lines 9-21 of '700, he explained the use of such magnetic pointers. It is described that “In the field of acupuncture of Chinese medicine, the application of magnetic treatment to an acupuncture point of human body is a new physical therapy. In clinical practice of magnetic acupuncture, a main acupuncture point or a plurality of acupuncture points shall be first picked up from human main and collateral channels which relate to the disease, and then a doctor or patient himself fixes the magnetic pole of the magnetic medical instrument to the acupuncture points to be treated. At present, the magnetic medical instruments mainly include magnetic medical instrument, magnets of slice or block shapes and magnetic needle etc.”

In the column 1, lines 16 to 22 of the U.S. Pat. No. 6,113,620, it is also described in that “In the Chinese medical art, acupuncture is a treatment for pain and illness in which thin needles are positioned just under the surface of the skin at special nerve centers (mediterian points) around the body.”

This means that there are certain points on the human body that the ‘magnet pointers’ are effective for healing certain diseases. Meanwhile most of the acupuncture needles available on the market are long slender rods of metal having a diameter less than 1 mm and the tip are very sharp to penetrate a human skin. Therefore, the above two prior arts are related with ‘magnet pointers’, not related with any ‘needle’.

It is the purpose of the current application to maximize the effect of magnetic fields to the human body. As reported by Professor James D. Livingston of MIT, the net magnetic field developed in the water is about 100,000 times smaller than the applied field. Though the strength of the magnet of the ‘pointer’ is 10,000 Gauss as described as the maximum strength in the '620, the net magnetic field of water in blood beneath the skin is 0.1 Gauss. That is weaker than the magnetic field strength of the Earth onto the human body, 0.5 Gauss. Therefore, the magnetic effect of the ‘pointer’ on a ‘mediterian point’ of a patient is doubtful.

Meanwhile, the magnetic field around a sharp needle end is very strong compared with the field around other parts of the needle. The acupuncture needle of the current application is applied onto any skin point of a patient (not only onto mediterian points), where the patient feels pain. The needle of the current application penetrates the skin of a patient and contacts with blood directly. Then the induced magnetic field on the tip of the needle facilitates blood circulation of a limited point of around the tip of the needle. It does not heal any disease. Instead, it removes the blue marks from a bruise by allowing the blood of abnormal formation align and pass through the capillaries to join the circulation of normal blood.

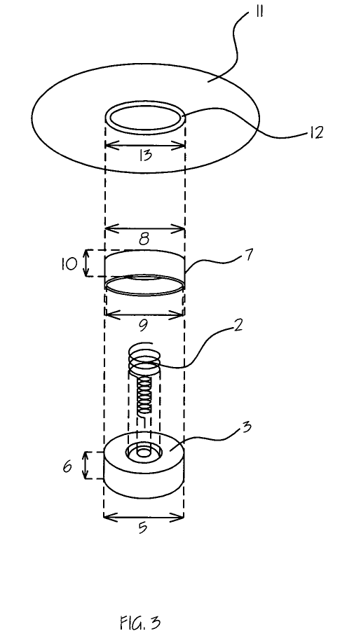

FIG. 3 is an exploded view of an acupuncture spring needle set (1) utilizing induced magnetic field of current application. The patch type acupuncture induced magnetic needle set (1) of the current application is comprised of a spring needle (2) of total height 3.1 mm and thickness 0.18 mm, an annular magnet (3) with inner-diameter (4) of 2.2 mm, outer diameter (5) of 7.0 mm, thickness (6) of 2.1 mm and has magnetic strength of 650 Gauss, a circular cap (7) of outer diameter (8) 7.5 mm, inner diameter (9) 7.3 mm and cap wall height (10) 2 mm, and an adhesive plaster (11) which has a round seat (12) of inner-diameter (13) of 7.5 mm for receiving the magnet cap.

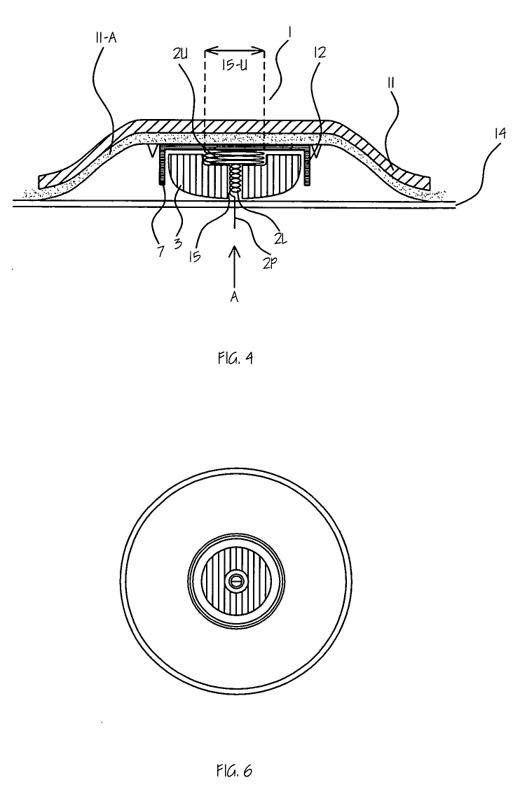

FIG. 4 is a cross-sectional view of an acupuncture spring needle set (1) with induced magnetic field of current application when attached to a skin (14) of a patient. The spring needle (2) is made of stainless steel STS 304 and coiled in two different diameters. Upper part (2-U) of the spring needle (2) is coiled in 3.4 mm diameter and lower part (2-L) of the spring needle is coiled in 2.0 mm. The last part (2-P) is straightened to form a needle. Total height of the coiled part of the spring needle is 2.1 mm, which is same as the thickness (6) of the annular magnet (3). The length (16) of the straight last part (2-P) is 1 mm. Therefore, the 1 mm of the last part (2-P) penetrates the skin (14) of a patient. The last part (2-P) is coated with Silver or Gold with 0.1 μM thickness. When a user patches the needle set (1) onto his/her skin, the 1 mm of the last part (2-P), straight needle, penetrate the user's skin.

The annular magnet (3) has a staged inner hole (15), the upper part diameter (15-U) 3.5 mm and the lower part diameter (inner-diameter) (4) is 2.2 mm. Such staged geometry of the inner hole (15) holds the spring needle (2) therein. The annular magnet (3) is adhered in a circular cap (7) that is made of steel. The adhesive plaster (11) holds the cap (7) in the round seat (12) that is the same as the adhesives (11-A) layer.

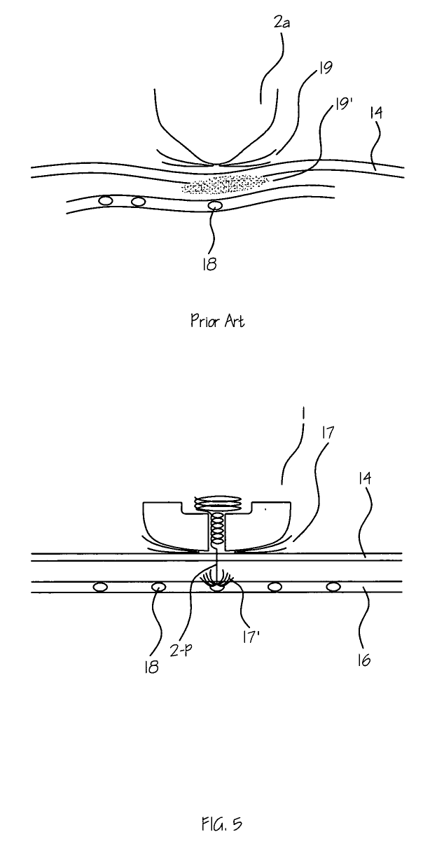

FIG. 5 is a schematic cross-sectional view comparing the magnetic field effect of the acupuncture spring needle utilizing the induced magnetic field of the current application and the magnetic pointer of the prior art when applied to the skin of a patient.

When a metal needle, which is not magnetic before, is adhered to a magnet, the metal needle also becomes a magnet, and the strength of the magnetic field is not decreased seriously throughout the needle. The strength of the magnetic field of the needle is the strongest at the tip. Similarly, the spring needle (2) becomes a magnet and the magnetic field is the strongest at the tip. When a patient patches the magnetic needle set (1) on the skin and presses, the last part (2-P) penetrates the skin (14) and may reach to a capillary blood vessel (16) close to the skin (14) of the patient while maintaining the similar magnetic field (17) strength. Then, the blood cells (18) are exposed to a magnetic field (17′) that is similar to that of the magnet (3).

Meanwhile, if the report of Professor James D. Livingston at M.I.T is right, the magnetic field (19) under the skin (14) of a patient using a magnet press (2a) of the prior art is about 1/100,000 of that of the magnet field (19′) of the magnet press (2a). Therefore, the influence of the magnetic field to a patient's blood of the current application is much more strong and effective.

FIG. 5 is a front view of an acupuncture spring needle set (1) utilizing induced magnetic field of current application seen from the view point ‘A’ in FIG. 4. The planar shape is a circle.

What is claimed is:

1. An acupuncture spring needle set, utilizing induced magnetic field, comprised of;
a stainless steel STS 304 spring needle, having a total height of 3.1 mm and thickness of 0.18 mm, and coiled in two different diameters, upper part of which is coiled in 3.4 mm diameter and lower part of which is coiled in 2.0 mm and the last part is straightened to form a needle having a total height of 2.1 mm for the coiled part and 1 mm for the straight last part that is coated with 0.1 μm thickness Gold to penetrate into the skin of a patient, and
an annular magnet with outer diameter of 7.0 mm and thickness of 2.1 mm and having a staged inner hole, whose upper part diameter is 3.5 mm and the lower part diameter is 2.2 mm, to receive the partially coiled needle therein, and has a magnetic strength of 650 Gauss, and
a metal circular cap, which receives the annular magnet with the spring needle therein, having outer diameter of 7.5 mm, inner diameter 7.3 mm and cap wall height 2 mm, and
an adhesive plaster which has a round seat of inner-diameter of 7.5 mm for receiving the magnet cap.

2. An acupuncture spring needle set of claim 1, wherein the straight last part of the needle is coated with 0.1 μm Silver.
